(19) United States
(12) Patent Application Publication
(10) Pub. No.: US 2025/0127625 A1
(43) Pub. Date: Apr. 24, 2025

(54) SPINAL IMPLANT DEVICE FOR MOTION PRESERVATION/DISC STABILIZATION

(71) Applicants: Ali Kiapour, Newton, MA (US); Matthew Shomper, Xenia, OH (US)

(72) Inventors: Ali Kiapour, Newton, MA (US); Matthew Shomper, Xenia, OH (US)

(21) Appl. No.: 18/919,725

(22) Filed: Oct. 18, 2024

Related U.S. Application Data
(60) Provisional application No. 63/544,910, filed on Oct. 19, 2023.

(51) Int. Cl. A61F 2/44; A61F 2/30

(52) U.S. Cl. CPC A61F 2/4425; A61F 2002/30904; A61F 2002/3093; A61F 2002/30952; A61F 2002/30985; A61F 2002/443

(57) ABSTRACT

A spinal implant device comprises a first plate configured to be disposed in proximity to a first vertebral body; a second plate configured to be disposed in proximity to a second vertebral body, the second vertebral body opposing the first vertebral body; and a lattice structure disposed between the first plate and the second plate, the lattice structure configured to adjust an instantaneous axis of rotation between the first plate and the second plate during relative movement of the first plate and the second plate.

### RELATED APPLICATIONS

This patent application claims the benefit of U.S. Provisional Application No. 63/544,910, filed on Oct. 19, 2023 and entitled “Patient-Specific 3D-Printed Motion Preservation/Dynamic Stabilizing Device for Spinal Motion Preservation/Disc Stabilization,” the contents and teachings of which are hereby incorporated by reference in their entirety.

### FIELD

Embodiments of the present innovation relate to devices used in orthopedic and spine surgery and specifically relate to devices used as a total joint replacement to replicate the mechanics of any intervertebral disc or spinal segment non-fixation stabilizer.

### BACKGROUND

A human spinal column includes three main regions: the cervical, thoracic, and lumbar spines. Each region includes functional spine units (FSU), which have one intervertebral disc and two adjacent vertebral bones. The intervertebral disc allows movement between adjacent vertebral bodies to absorb shock and transmit loads through the spinal column.

Spinal disease can affect the functionality of FSUs and can typically be caused by a change in the structure of a healthy intervertebral disk. Further, in severe cases, spinal disease can cause a healthy disk to herniate, which creates extra pressure exerted on the spinal cord, thereby resulting in back pain or neck pain.

One conventional treatment for spinal disease includes fixation, whereby a surgeon fills and spaces the damaged disc with interbody spacers/cages and fixates and immobilizes the damaged FSU. In this treatment of spinal disorders (e.g., spinal fixation or fusion), metallic/non-metallic screws, hooks, rods, plates, nails, anchors, and spacers are used for immobilization of the damaged spinal segment, the ultimate goal being a permanent bony fusion.

Alternately, total disc replacement (TDR) devices can be utilized to preserve motion in the surgically treated spinal segment, to eliminate many of the shortcomings of traditional fusion or interbody fixation techniques. Total disc replacement (TDR) is a surgical procedure used to treat degenerative disc disease and other conditions affecting the spine. The procedure involves removing a damaged intervertebral disc and replacing it with an artificial one, with the goal of restoring normal disc function while preserving motion in the affected segment of the spine. Unlike spinal fusion, which immobilizes the affected area, TDR maintains mobility, potentially reducing the stress on adjacent spinal segments and lowering the risk of future degeneration in those areas. TDR is commonly performed in the cervical and lumbar regions, offering a less invasive alternative with faster recovery times and improved quality of life for patients experiencing chronic back or neck pain due to disc degeneration.

### SUMMARY

Conventional spinal disease treatment options can suffer from a variety of deficiencies. For example, traditional surgical treatment options using fixation often result in long-term complications, such as adjacent segment disease or degeneration of the intervertebral disc tissue due to excess load applied to the FSUs adjacent to the fixation location due to compromised motion and biomechanics. Further, conventional TDR systems are manufactured as either mechanical structures with articulating metallic and polymeric components or elastomeric structures with a deformable material sandwiched between two metallic endplates. Each of the technologies suffer from limitations, such as wear and tear of the polymeric components and degradation of the elastomeric components, which can jeopardize the long-term clinical outcomes of TDR procedures.

Additionally, the existing, conventional disc replacement solutions have pre-defined biomechanical characteristics with pre-designed motion ranges and characteristics and are made based on one-design fits all basis. As provided above, conventional TDR systems include rigid mechanical components or deformable elastomer/polymer components where the displacement or deformation of the materials defines the angular motion in the TDR implant. While these systems are configured to provide flexibility and range of motion close to that of spine, the systems fail to account for the quality of motion and transition of instantaneous axis of rotation from one extreme to the other extreme over the entire range of motion of the FSU.

By contrast to conventional spinal disease treatment options, embodiments of the present innovation relate to a spinal implant device for spinal motion preservation/disc stabilization. In one arrangement, the spinal implant device can be custom-designed and manufactured to match the particular mechanics of a patient's intervertebral disc and to preserve the anatomical motion of the spine during anatomical loadings or provide controlled segmental kinematics to stabilize the treated intervertebral discs. The device can be used for disc replacement/stabilizer at any level in the spine (e.g., cervical, thoracic, and lumbar).

In one arrangement, the innovation relates to a spinal implant device, comprising a first plate configured to be disposed in proximity to a first vertebral body; a second plate configured to be disposed in proximity to a second vertebral body, the second vertebral body opposing the first vertebral body; and a lattice structure disposed between the first plate and the second plate, the lattice structure configured to adjust an instantaneous axis of rotation between the first plate and the second plate during relative movement of the first plate and the second plate.

In one arrangement, the innovation relates to, in a spinal implant device production system, a method for generating a spinal implant device, comprising: receiving load characteristics and geometric characteristics associated with a functional spine unit; selecting a spinal implant device template from a set of templates based upon the load characteristics; identifying instantaneous axis of rotation values for the functional spine unit based upon the geometric characteristics; providing the spinal implant device template and the instantaneous axis of rotation values to a topology optimization engine to generate a spinal implant device model; and printing the spinal implant device based upon the spinal implant device model.

In one arrangement, the innovation relates to a spinal implant device production system, comprising: an additive manufacturing platform having a controller comprising a processor and memory, the controller configured to: receive load characteristics and geometric characteristics associated with a functional spine unit, select a spinal implant device template from a set of templates based upon the load characteristics, identify the instantaneous axis of rotation values for the functional spine unit based upon the geometric characteristics, provide the spinal implant device template and the instantaneous axis of rotation values to a topology optimization engine to generate a spinal implant device model, and an additive manufacturing device disposed in electrical communication with the additive manufacturing platform, the additive manufacturing device configured to print the spinal implant device based upon the spinal implant device model.

### BRIEF DESCRIPTION OF THE DRAWINGS

The foregoing and other objects, features and advantages will be apparent from the following description of particular embodiments of the innovation, as illustrated in the accompanying drawings in which like reference characters refer to the same parts throughout the different views. The drawings are not necessarily to scale, emphasis instead being placed upon illustrating the principles of various embodiments of the innovation.

FIG. 1 illustrates an example arrangement of a spinal implant device, according to one arrangement.

FIG. 2A illustrates an instantaneous axis of rotation of a functional spine unit.

FIG. 2B illustrates flexion of the functional spine unit of FIG. 2A.

FIG. 2C illustrates extension of the functional spine unit of FIG. 2A.

FIG. 2D illustrates a change in the instantaneous axis of rotation of the functional spine unit of FIG. 2A between flexion and extension.

FIG. 3A illustrates the spinal implant device of FIG. 1 deformed under an anatomical load and mirroring the instantaneous axis of rotation of the functional spine unit in extension, according to one arrangement.

FIG. 3B illustrates the spinal implant device of FIG. 1 deformed under an anatomical load and mirroring the instantaneous axis of rotation of the functional spine unit in flexion, according to one arrangement.

FIG. 4 illustrates a normal load-displacement curve of a functional spine unit in flexion-extension motion.

FIG. 5A illustrates the spinal implant device of FIG. 1 having a coil-based lattice, according to one arrangement.

FIG. 5B illustrates the spinal implant device of FIG. 1 having an auxetic-based lattice, according to one arrangement.

FIG. 5C illustrates the spinal implant device of FIG. 1 having a beam-based lattice, according to one arrangement.

FIG. 6 illustrates an example of the spinal implant device of FIG. 1 having a lattice structure configured with a varying lattice structure density, according to one arrangement.

FIG. 7 illustrates an example of the spinal implant device of FIG. 1 having a lattice structure configured with a varying lattice structure stiffness, according to one arrangement.

FIG. 8 illustrates an example of the spinal implant device of FIG. 1 having a lattice structure configured with a varying functionally-graded thickness, according to one arrangement.

FIG. 9 illustrates an example of the spinal implant device of FIG. 1 having a lattice structure configured with varying functionally graded morphology, according to one arrangement.

FIG. 10A illustrates a front view of the spinal implant device of FIG. 1 having a fixation mechanism, according to one arrangement.

FIG. 10B illustrates a top perspective view of the spinal implant device of FIG. 10A, according to one arrangement.

FIG. 11 illustrates a side sectional view of the spinal implant of FIG. 1 having a fixation mechanism that includes an osseointegration element, according to one arrangement.

FIG. 12 illustrates a schematic representation of a spinal implant device production system, according to one arrangement.

FIG. 13 is a flowchart of a process performed by the spinal implant device production system of FIG. 8 when generating a spinal implant device, according to one arrangement.

FIG. 14 illustrates an iterative design process performed by the spinal implant device production system, according to one arrangement.

### DETAILED DESCRIPTION

Embodiments of the present innovation relate to a spinal implant device for spinal motion preservation/disc stabilization. In one arrangement, the spinal implant device can be custom designed and manufactured to match the particular mechanics of a patient's intervertebral disc and to preserve the anatomical motion of the spine during anatomical loadings or provide controlled segmental kinematics to stabilize the treated intervertebral discs. The device can be used for disc replacement/stabilizer at any level in the spine (e.g., cervical, thoracic, and lumbar).

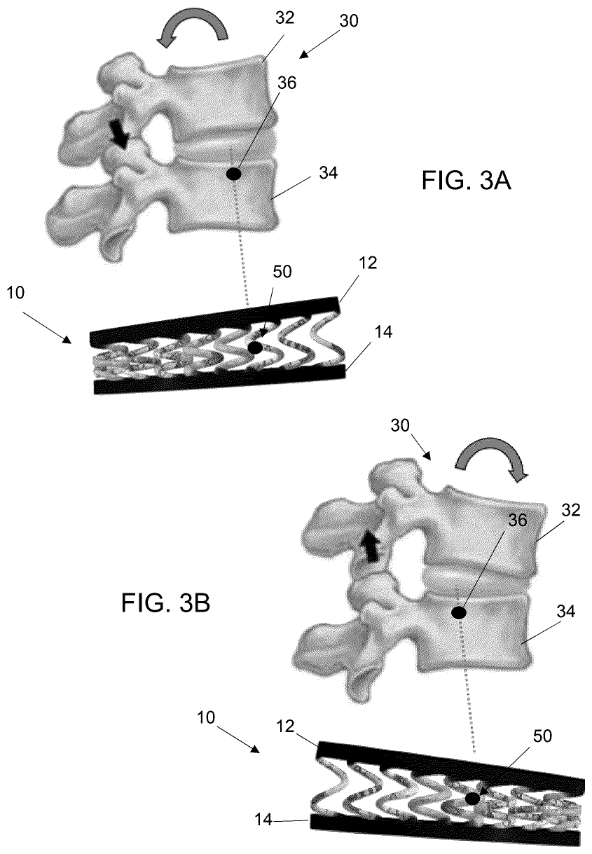

FIG. 3 illustrates an example arrangement of a spinal implant device 10, such as a lumbar disc replacement device, which is configured to deliver the general biomechanics of spinal disc systems (e.g., lumbar, cervical, and thoracic) within a spine 18. In one arrangement, the spinal implant device 10 can be configured as a non-fixation device that provides motion stabilization to the spine 18 under different anatomical motions. For example, with such a configuration, the spinal implant device 10 can limit the maximum range of motion in any direction (e.g., anterior-posterior, medial-lateral, and axial rotation) and with respect to any plane ad with respect to any specific biomechanical relevant values. Alternatively, in one arrangement, the spinal implant device 10 can be configured as a motion preservation device, such as in a total disc replacement, to mimic the kinematics of a spinal disc under anatomical loads.

The spinal implant device 10 includes a variety of components. For example, the spinal implant device 10 can include a first plate 12, a second plate 14, and a lattice structure 16 disposed between the first plate 12 and the second plate 14.

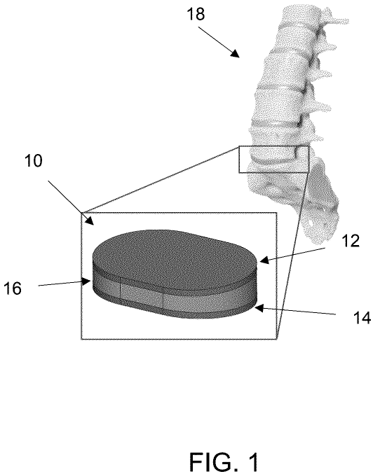

The first plate 12 of the spinal implant device 10 is configured to be disposed in proximity to a first vertebral body 20 while the second plate 14 is configured to be disposed in proximity to a second, opposing vertebral body 22. For example, as illustrated in FIG. 1, the spinal implant device 10 is configured as an L5-S1 replacement disc. As such, the first plate 12 of the spinal implant device 10 is disposed against the L5 vertebra 20 while the second plate 14 of the spinal implant device 10 10 is disposed against the S1 vertebra 22. With such positioning, the first and second plates 12, 14 are configured to transmit the loads received from the respective vertebra 20, 22 to the lattice structure 16.

In one arrangement, the first plate 12 and the second plate 14 can be configured with a variety of surface geometries. For example, each one of, or either, the first plate 12 and the second plate 14 can have a surface geometry that is planar (e.g., angled or parallel), curved in a single axis, bi-convex curved, or configured as a series of complex splines or surfaces that conform to the local anatomy of the adjacent vertebral bone surfaces.

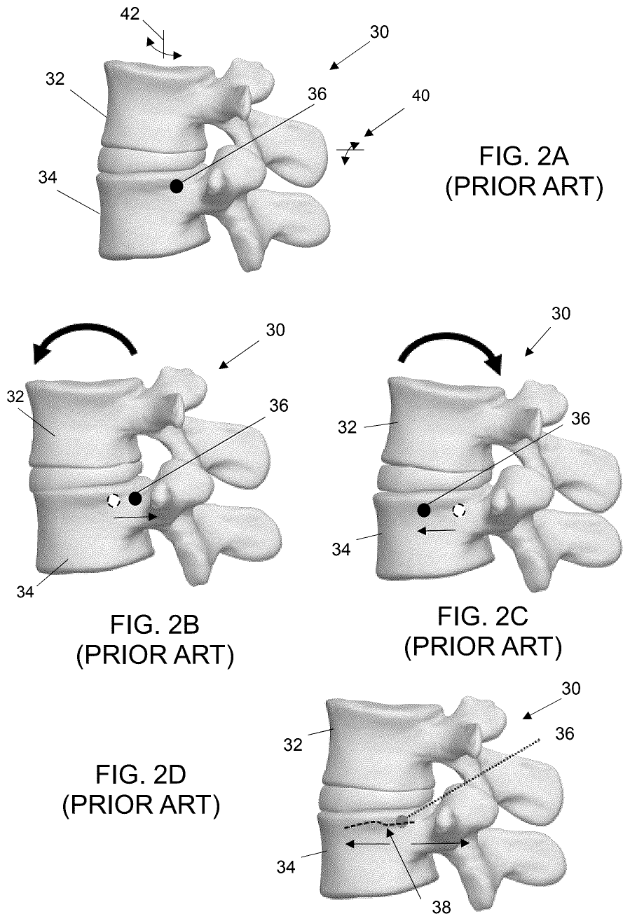

The lattice structure 16 is configured to provide flexibility and range of motion similar to that of a relatively healthy functional spine unit. With reference to FIG. 2A, a functional spine unit 30 defines an instantaneous center of rotation 36 in a neutral position. During relative motion of the adjacent vertebrae 32, 34, the location of the instantaneous center of rotation 36 changes relative to the instantaneous center of rotation 36 in the neutral position. For example, in the case where the adjacent vertebrae 32, 34 of the functional spine unit 30 rotate along an anterior direction (e.g., in flexion as shown in FIG. 2B) within the sagittal plane, the instantaneous center of rotation 36 moves toward a posterior location of the functional spine unit 30. Further, in the case where the adjacent vertebrae 32, 34 of the functional spine unit 30 rotate along a posterior direction (e.g., in extension as shown in FIG. 2C) within the sagittal plane, the instantaneous center of rotation 36 moves toward an anterior location of the functional spine unit 30. As such, as indicated in FIG. 2D, the instantaneous center of rotation 36 changes position 38 over the course of full flexion and extension. Further, it is noted that the location of the instantaneous center of rotation 36 changes relative to the neutral position in the case where the adjacent vertebrae 32, 34 of the functional spine unit 30 rotate along a medial-lateral direction 40 within a coronal plane or about a longitudinal axis 42 within a transverse plane.

As indicated in FIGS. 3A and 3B, the lattice structure 16 is configured to position the location of the instantaneous center of rotation 50 of the spinal implant device 10 such that the instantaneous center of rotation 50 substantially corresponds to the anatomical location of the instantaneous center of rotation 36 of the functional spine unit 30. For example, with reference to FIG. 3A, the lattice structure 16 is configured to position the location of the instantaneous center of rotation 50 of the spinal implant device 10 (e.g., between the first plate 12 and the second plate 14) to correspond to the anatomical location of the instantaneous center of rotation 36 of the functional spine unit 30 during extension in the sagittal plane. With further reference to FIG. 3B, the lattice structure 16 is configured to position the location of the instantaneous center of rotation 50 of the spinal implant device 10 (e.g., between the first plate 12 and the second plate 14) to correspond to the anatomical location of the instantaneous center of rotation 36 of the functional spine unit 30 during flexion in the sagittal plane. While not shown, the lattice structure 16 is also configured to position the location of the instantaneous center of rotation 50 of the spinal implant device 10 (e.g., between the first plate 12 and the second plate 14) to correspond with the anatomical location of the instantaneous center of rotation 36 of the functional spine unit 30 in the case where the adjacent vertebrae 32, 34 of the functional spine unit 30 rotate along a medial-lateral direction within a coronal plane and in the case where the adjacent vertebrae 32, 34 of the functional spine unit 30 rotate about a longitudinal axis within a transverse plane. Accordingly, the lattice structure 16 provides the spinal implant device 10 with the ability to mimic the quantity of motion (e.g., the anatomical range of motion) of a functional spine unit 30.

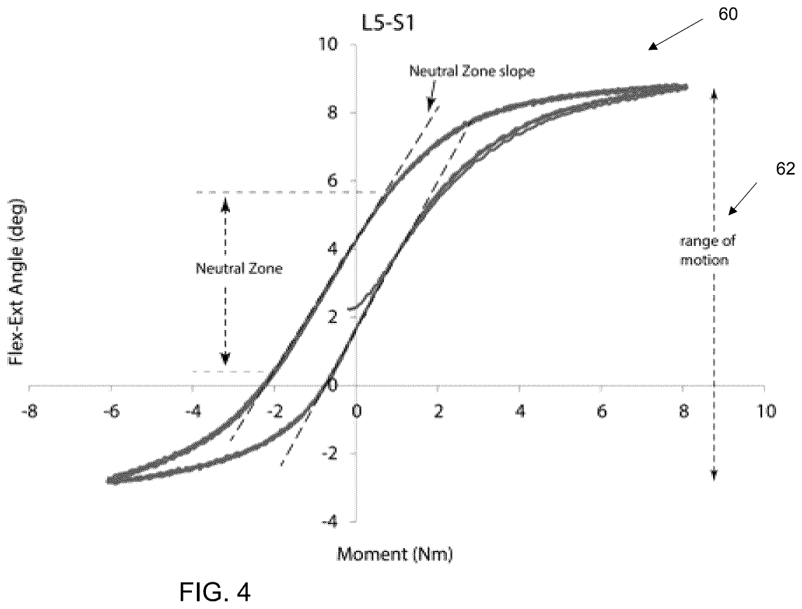

Further, the lattice structure 16 is configured to provide the spinal implant device 10 with the ability to mimic the quality of motion of functional spine unit 30. For example, with reference to FIGS. 3A and 3B, the spinal implant device 10 can be configured to deform to provide controlled segmental motion or to replicate the motion of the functional spine unit 30 under anatomical load. As such, and as will be described in detail below, the spinal implant device 10 can be configured to provide a unique and pre-specified quality of motion. For example, as indicated in the load-displacement curve 60 of FIG. 4, the spinal implant device 10 can be configured to deliver a non-linear and variable mechanical characteristics and range of motion 62 at different load rates which correspond to that of a functional spine unit in flexion-extension motion.

In one arrangement, in order to provide the spinal implant device 10 with the ability to mimic the quantity of motion and the quality of motion of a functional spine unit 30, the lattice structure 16 can include a variety of mechanical and geometric configurations, examples of which are provided as follows.

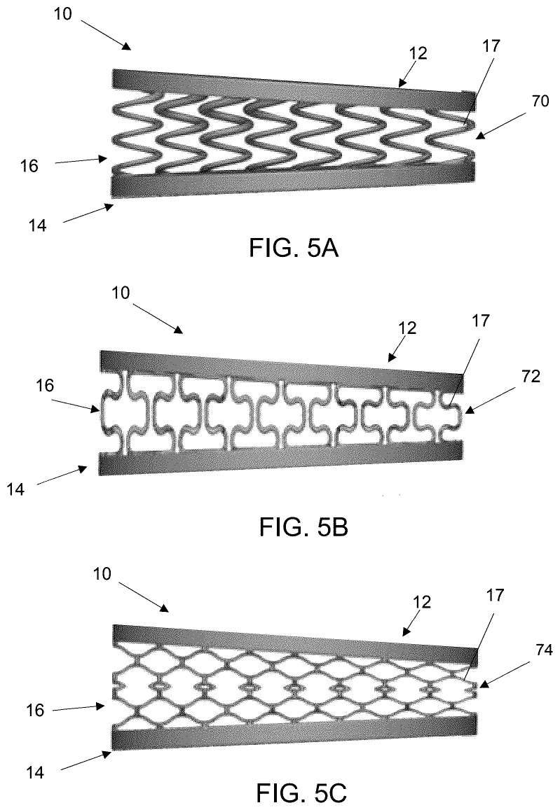

In one arrangement, the lattice structure 16 can be configured with a variety of cell geometries. For example, as shown in FIGS. 5A-5C, the lattice structure 16 can be configured as a coil structure 70, as an auxetic structure 72, or as a beam-based structure 74. Further, the lattice structure 16 can be configured with a particular volume fraction, cell size, strut width, and/or nodal connection points, for example. In one arrangement, the lattice structure 16 can include regularly repeating (e.g., tesselating) lattice strands, as shown in FIGS. 5A-5C or stochastic (e.g., randomly distributed) lattice strands.

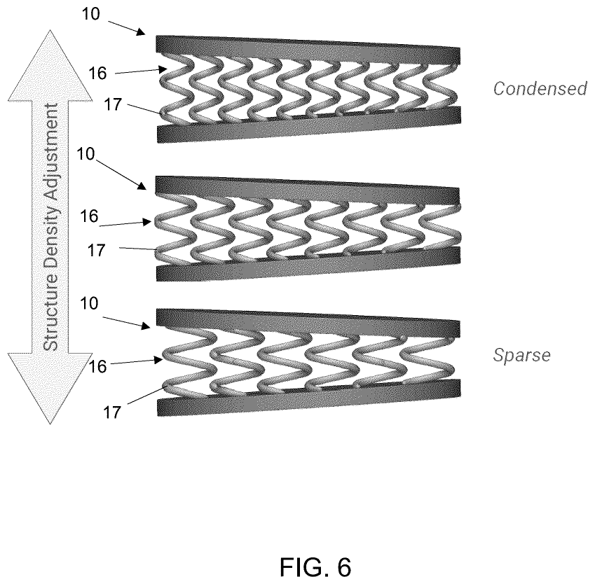

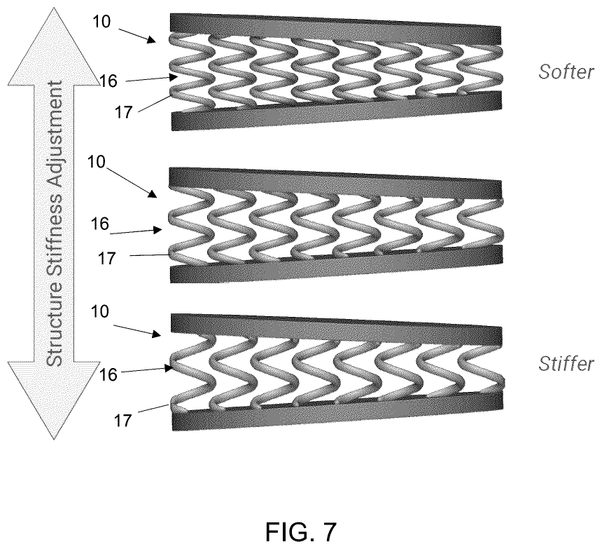

In one arrangement, with reference to FIG. 6, the lattice structure 16 can be configured with a variety of lattice strand densities, ranging from a relatively condensed arrangement to a relatively sparse arrangement. In one arrangement, with reference to FIG. 7, the lattice structure 16 can be configured with a variety of lattice strand stiffness values, ranging from relatively softer or low stiffness to a relatively high stiffness.

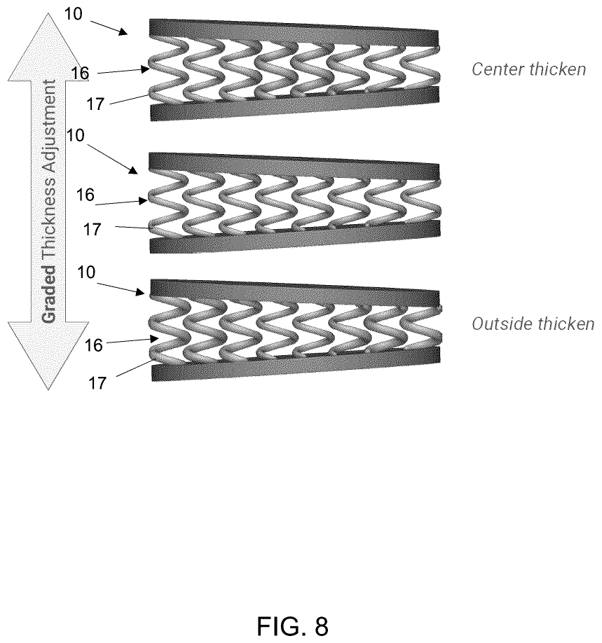

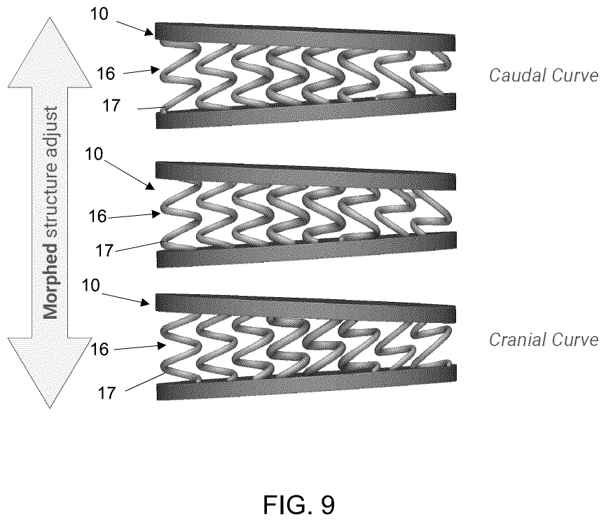

In one arrangement, with reference to FIG. 8, the lattice structure 16 can be configured as a functionally graded structure, ranging from relatively thicker lattice strands 17 disposed at the center of the spinal implant device 10 to relatively thicker lattice strands 17 disposed at the outer periphery of the spinal implant device 10. In one arrangement, with reference to FIG. 9, the lattice structure 16 can be configured as having a functionally graded morphology, ranging from lattice strands 17 defining caudal curves to lattice strands 17 defining cranial curves.

As provided above, in use, the first plate 12 of the spinal implant device 10 is configured to be disposed in proximity to a first vertebral body 20 while the second plate 14 is configured to be disposed in proximity to a second, opposing vertebral body 22. In one arrangement, the spinal implant device 10 can include a fixation mechanism configured to secure the spinal implant device 10 to the vertebral bodies 20, 22.

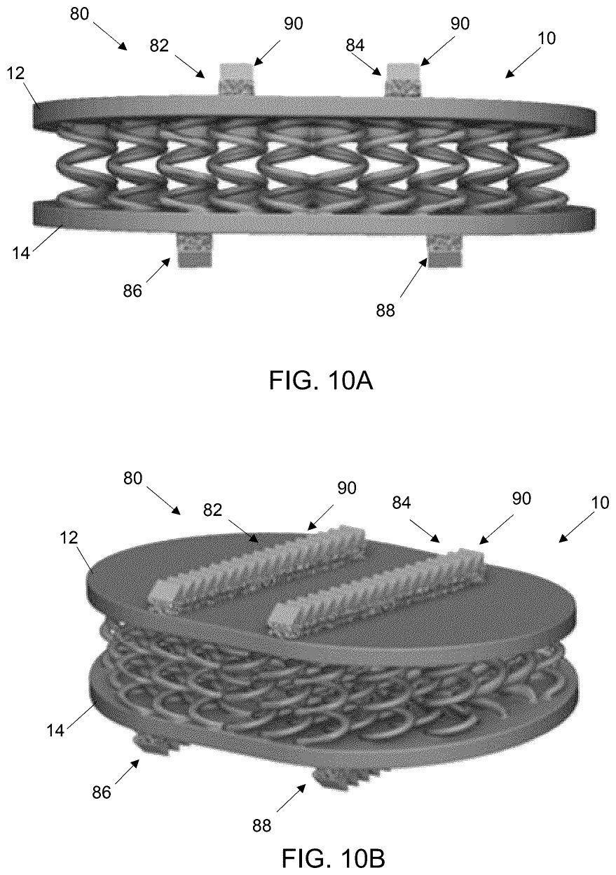

In one arrangement, with reference to FIGS. 10A and 10B, the spinal implant device 10 includes a fixation mechanism 80 extending from the first plate 12 and the second plate 14 toward opposing vertebral bodies 20, 22 (not shown). As illustrated, the fixation mechanism 80 can include a keel mechanism defining a series of protrusions. For example, the fixation mechanism 80 includes a first and second keel mechanism 82, 84 extending from the first plate 12 and first and second keel mechanism 86, 88 extending from the second plate 14. The protrusions 90 extending from the first and second plates 12, 14 are configured to engage the bony endplates of the corresponding vertebral bodies 20, 22 to couple the spinal implant device 10 to the vertebral bodies 20, 22, thereby incorporating the spinal implant device 10 as part of a functional spine unit.

As shown, each plate 12, 14 includes two respective keel mechanisms. As such, the spinal implant device 10 is configured for use within a patient's lumbar spine region. However, each plate 12, 14 can include any number of keel mechanisms. For example, each plate 12, 14 can be configured to include a single keel mechanism. With such a configuration, the spinal implant device 10 can be utilized within a neck region of a patient.

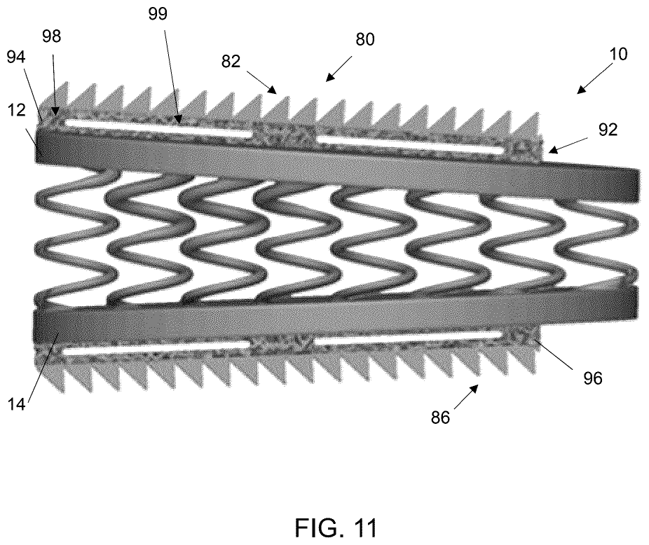

In one arrangement, the fixation mechanism 80 can also include an osscointegration mechanism 92 which is configured to provide osseointegration of the spinal implant device 10 relative to a respective vertebral body 20, 22. For example, with reference to FIG. 11, osscointegration mechanism 92 includes an osseointegration element 92 disposed between the first keel mechanism 82 and the first plate 12 and includes an osscointegration element 94 disposed between the first keel mechanism 86 and the second plate 14. The osscointegration mechanism 92 can be configured in a variety of ways. For example, the osscointegration mechanism 92 can be configured as a macro-lattice structure 98 or as defining one or more pockets 99 which allow for bony ingrowth to occur relative to the spinal implant device 10.

The spinal implant device 10 can be manufactured from a variety of materials using a variety of manufacturing processes. In one arrangement, the spinal implant device 10 is manufactured from an additive manufacturing material using an additive printing (e.g., 3D-printed) process. For example, any of the first plate 12, the second plate 14, the lattice structure 16, and the fixation mechanism 80 can be 3D-printed from a metal material, such as titanium or Nitinol, or from a non-metallic material, such as a polymer and/or a plastic material. Further, the device 10 can be manufactured using a variety of manufacturing techniques. For example, the first plate 12, the second plate 14, and the lattice structure 16 can be 3D-printed as a one-piece part or 3D-printed as separate components 12, 14, 16 and later assembled to form the spinal implant device 10. In the case where the spinal implant device 10 is assembled from separate components, different (i.e., dissimilar) materials can be utilized for the separate components 12, 14, 16 (e.g., a polymer core lattice structure 16 with metal end plates 12, 14).

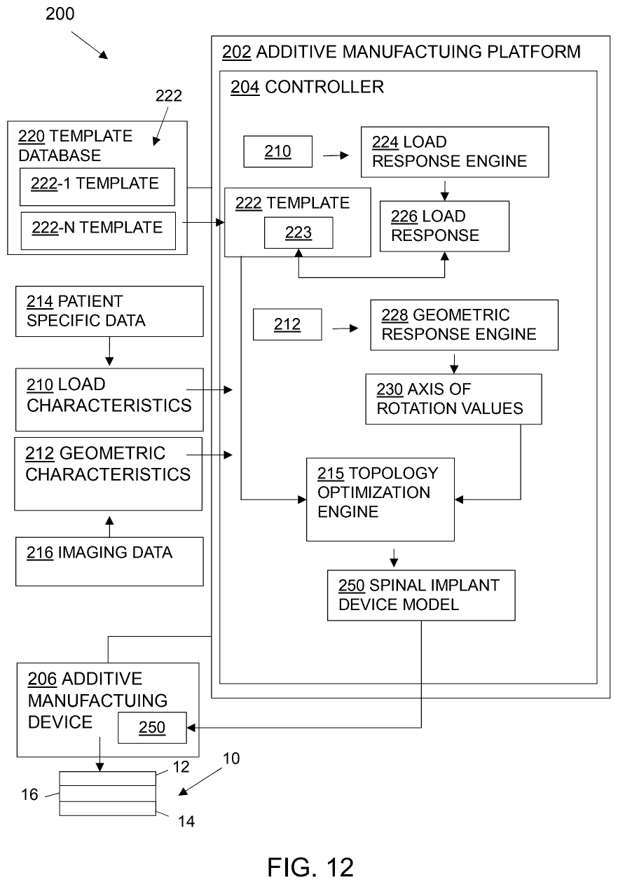

In one arrangement, the spinal implant device 10 can be designed and manufactured to match any patient's unique functional spine unit biomechanical parameters. For example, FIG. 12 illustrates a schematic representation of a spinal implant device production system 200 which is configured to develop and print a spinal implant device 10 having particular biomechanical parameters associated with a particular patient. The resulting spinal implant device 10 includes a lattice structure 16 that is configured to adjust the instantaneous axis of rotation between the first plate 12 and the second plate 14 according to patient data associated with a patient-specific functional spine unit (i.e., as associated with a particular patient).

In one arrangement, the production system 200 can include an additive manufacturing platform 202, such as a computerized device having a controller 206 (e.g., a memory and processor), configured to design the lattice structure 16 of a spinal implant device 10 based upon particular patient data 208. For example, the controller 206 is configured to execute a topology optimization engine 215 to generate a spinal implant device model 250 used to generate the spinal implant device 10, as will be described in detail below.

The spinal implant device production system 200 also includes an additive manufacturing device 206, such as a 3D printing device, disposed in electrical communication, such as a local area network (LAN) or a wide area network (WAN), with the additive manufacturing platform 202. The additive manufacturing platform 202 can provide the design of the spinal implant device 10 to the additive manufacturing device 206 for printing. As such, the spinal implant device production system 200 can generate a spinal implant device 10 which is customized as a patient-specific implant having a lattice structure 16 configured to match a particular anatomical range (e.g., quantity) of motion and quality of motion associated with the patient's functional spine unit.

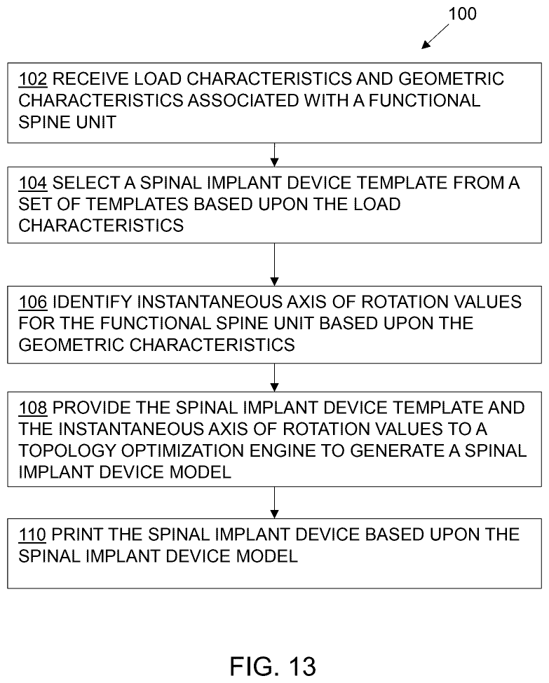

FIG. 13 is a flowchart 100 of a process performed by the spinal implant device production system 200 when generating a spinal implant device 10, according to one arrangement.

In element 102, the spinal implant device production system 200 receives load characteristics 210 and geometric characteristics 212 associated with a functional spine unit. In one arrangement, at the beginning of the process, a system operator collects the load characteristics 210 and geometric characteristics 212 from one or more functional spine units of a patient (e.g., a functional spine unit which causes pain in the patient) and provides the characteristics 210, 212 to the additive manufacturing platform 202 for further processing.

In one arrangement, the system operator can collect patient specific data 214 associated with one or more functional spine units of the patient as the load characteristics 210 for the patient. For example, the patient specific data 214 of a functional spine unit can be based upon the patient's weight, anatomy, required range of motion, load conditions, and/or any other characteristics (e.g., age, spinal health, etc.). As such, the system operator can provide the patient specific data 214 to the additive manufacturing platform 202 as the load characteristics 210.

In one arrangement, and with continued reference to FIG. 12, the system operator can collect imaging data 216 identifying the geometry and/or motion of one or more functional spine units of the patient as the geometric characteristics 212. The imaging data 216 can be collected in a variety of ways.

In one example, the system operator can utilize computed tomography (CT) imaging to collect imaging data 216 that identifies a variety of metrics associated with one or more of the patient's functional spinal units. For example, the imaging data 126 can include spine posture of the patient in the sagittal plane at extreme ranges of motion (e.g., flexion and extension), the quality of motion, the non-linear motion characteristics, functional spinal unit height data, and functional spinal unit width data for one or more of the patient's functional spine units. The system operator can provide the imaging data 216 to the additive manufacturing platform 202 as the geometric characteristics 212 associated with the patient's functional spine units.

In another example, the system operator can utilize open magnetic resonance imaging (MRI) to collect imaging data 216, such as video, that identifies the motion of one or more functional spine units of the patient. The imaging data 126 can further include functional spinal unit height data, and functional spinal unit width data for one or more of the patient's functional spine units, for example. The system operator can provide the imaging data 126 to the additive manufacturing platform 202 as the geometric characteristics 212 associated with the patient's functional spine units.

In another example, the system operator can utilize Vertebral Motion Analysis (VMA) or Digital Motion X-ray (DMX) to capture images of the motion of a patient's functional spine unit and can provide these images to a design platform for assessment of the motion using an artificial intelligence (AI) algorithm and extraction of trajectory of the instantaneous axis of rotation 36.

Returning to FIG. 13, in clement 104, the spinal implant device production system 200 selects a spinal implant device template 222 from a set of templates 220 based upon the load characteristics 210. In one arrangement, with reference to FIG. 12, the additive manufacturing platform 202 is disposed in electrical communication with a spinal implant device template database 220 which stores a variety of preconfigured spinal implant device template 222-1 through 222-N. Each spinal implant device template 222-1 represents a simulated spinal implant device having a lattice structure configured with a particular load response 223, such as a modulus of elasticity or stiffness value.

In order to select a particular spinal implant device template 222-1 through 222-N as the basis for development of the design of a spinal implant device 10 based upon the load characteristics 210 of a particular patient, the additive manufacturing platform 202 is configured to execute a load response engine 224. With such execution, the load response engine 224 receives the load characteristics 210 (e.g., patient's weight, age, spinal health, etc.) and, based upon the load characteristics 210, identifies a load response 226, such as the modulus of elasticity or stiffness, of one or more of the patient's functional spine units. Next, the additive manufacturing platform 202 can review the spinal implant device template 222-1 through 222-N of the template database 220 to identify a spinal implant device template 222 having a load response 223 which corresponds to or approaches (e.g., is approximate to) the load response 226 of a particular functional spine unit of the patient. When such a correspondence or proximity exists, the additive manufacturing platform 202 can select that spinal implant device template 222 for further processing and development.

Returning to FIG. 13, in element 106, the spinal implant device production system 200 identifies instantaneous axis of rotation values 230 for the functional spine unit based upon the geometric characteristics 212.

In one arrangement, with reference to FIG. 12, the additive manufacturing platform 202 is configured to execute a geometric response engine 228, such as an artificial intelligence-based engine, to determine the instantaneous axis of rotation values for the patient's functional spine unit. For example, during operation the geometric response engine 228 receives the geometric characteristics 212 (e.g., imaging data) from the spinal implant device production system 200 and is configured to perform an image segmentation on the geometric characteristics 212 to identify the patient's functional spine unit anatomy on the geometric characteristics 212 to perform an extraction of the biomechanical details from the geometric characteristics 212 to generate a 3D model of the patient's functional spine unit. Based upon the anatomic and biomechanical data retrieved from the geometric characteristics 212, the geometric response engine 228 is further configured to analyze the geometric characteristics 212 and extract a range of motion of the patient's functional spinal unit and a pattern of the motion, such as the instantaneous axis of rotation values 230 for the patient's functional spinal unit.

Returning to FIG. 13, in element 108, the spinal implant device production system 200 provides the spinal implant device template 222 and the instantaneous axis of rotation values 230 to a topology optimization engine 215 to generate a spinal implant device model 250. In one arrangement, with reference to FIG. 12, during the process of topology optimization, the topology optimization engine 215 is configured to optimize the lattice structure of the spinal implant device template 222 based upon the displacement requirements identified for the patient's functional spine unit, as defined by the instantaneous axis of rotation values 230.

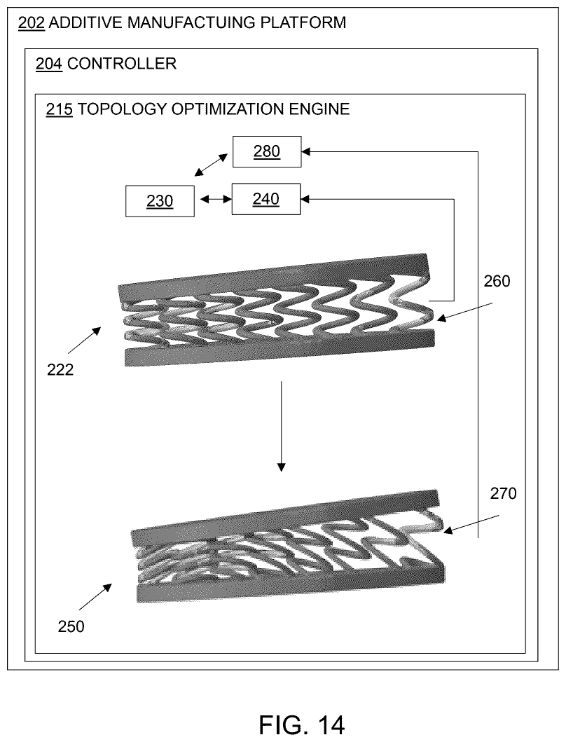

For example, with additional reference to FIG. 14, the topology optimization engine 215 is configured to execute a displacement simulation on the spinal implant device template 222 using the patient-specific load characteristics 210 (e.g., patient weight, anatomy, required range of motion, and load conditions) and to identify the instantaneous axis of rotation values 240 resulting from the simulation. The topology optimization engine 215 can then compare the instantaneous axis of rotation values 240 of the spinal implant device template 122 with the instantaneous axis of rotation values 230 for the functional spine unit. In the case where the topology optimization engine 215 identifies a lack of correspondence (e.g., the values 230, 240 are unequal to or do not approach each other) between the instantaneous axis of rotation values 230 for the functional spine unit and the instantaneous axis of rotation values 240 of the spinal implant device template 222, the topology optimization engine 215 is configured to adjust a lattice metric 260 of the spinal implant device template 222. For example, the topology optimization engine 215 can adjust the structure and layout of the lattice spinal implant device template 222, such as provided in FIGS. 5A-9 above (e.g., adjust thickness, density, and mechanical properties of the lattice metric 260 in different areas), in order to mimic the quantity of motion and the quality of motion of the patient's functional spine unit. The topology optimization engine 215 can then generate a spinal implant device model 250 having the adjusted lattice metric 270 of the spinal implant device template 222.

The topology optimization engine 215 is configured to then execute a displacement simulation on the spinal implant device model 250 using the patient-specific load characteristics 210 and to identify the instantaneous axis of rotation values 280 resulting from the simulation. The topology optimization engine 215 can continue through the process of comparing the values 280 with the instantaneous axis of rotation values 230 for the functional spine unit, adjusting the lattice metrics 270 of the spinal implant device model 250 to iteratively generate subsequent spinal implant device models 300 until the resulting spinal implant device model 250 has a motion quality and center of rotation positions that mimic that of the patient's functional spine unit.

Returning to FIG. 12, in element 110, the spinal implant device production system 200 is configured to print the spinal implant device 10 based upon the spinal implant device model 250. In one arrangement, with reference to FIG. 12, additive manufacturing platform 202 can provide the spinal implant device model 250 to the additive manufacturing device 206 which utilizes the spinal implant device model 250 as instructions to generate the spinal implant device 10. Alternately, the additive manufacturing platform 202 utilizes the spinal implant device model 250 to direct the additive manufacturing device 206 to generate a device 10 having equivalent characteristics. For example, the additive manufacturing device 206 can print a first plate 12, a second plate, and a lattice structure 16 that are designed to provoke specific stress/strain/displacement responses and instantaneous axis of rotation values as identified in the functional spine unit of the patient. As such, the system 200 can provide a spinal implant device 10 having a customizable lattice structure that provided flexibility and range of motion similar to that of a normal spine.

As described above, the spinal implant device production system 200 is configured to generate a spinal implant device 10 based upon the functional spine unit of a specific patient. Such a description is by way of example only. In one arrangement, the spinal implant device production system 200 is configured to generate a generic, non-patient-specific spinal implant device 10. For example, the system 200 can generate and optimize a generic spinal implant model based upon known, general biomechanical data for the human spine. In one arrangement, the spinal implant device production system 200 can utilize artificial intelligence and machine learning algorithms to optimize the design of the generic spinal implant model using clinical data from a group of subjects.

While various embodiments of the innovation have been particularly shown and described, it will be understood by those skilled in the art that various changes in form and details may be made therein without departing from the spirit and scope of the innovation as defined by the appended claims.

What is claimed is:

1. A spinal implant device, comprising:
a first plate configured to be disposed in proximity to a first vertebral body;
a second plate configured to be disposed in proximity to a second vertebral body, the second vertebral body opposing the first vertebral body; and
a lattice structure disposed between the first plate and the second plate, the lattice structure configured to adjust an instantaneous axis of rotation between the first plate and the second plate during relative movement of the first plate and the second plate.

2. The spinal implant device of claim 1, wherein each of the first plate, the second plate, and the lattice structure are formed from an additive manufacturing material.

3. The spinal implant device of claim 1, wherein the lattice structure is configured to adjust the instantaneous axis of rotation between the first plate and the second plate during at least one of relative anterior-posterior movement of the first plate and the second plate, relative medial-lateral movement of the first plate and the second plate, and relative rotational movement of the first plate and the second plate.

4. The spinal implant device of claim 1, wherein the lattice structure is configured to adjust the instantaneous axis of rotation between the first plate and the second plate according to patient data associated with a patient-specific functional spine unit.

5. The spinal implant device of claim 1, wherein at least one of the first plate and the second plate comprises a fixation mechanism configured to couple the at least one of the first plate and the second plate to an opposing vertebral body.

6. The spinal implant device of claim 4, wherein the fixation mechanism comprises a keel mechanism defining a series of protrusions extending from the at least one of the first plate and the second plate toward the opposing vertebral body.

7. The spinal implant device of claim 4, wherein the fixation mechanism further comprises an osseointegration element disposed between the keel mechanism and at least one of the first plate and the second plate.

8. In a spinal implant device production system, a method for generating a spinal implant device, comprising:
receiving load characteristics and geometric characteristics associated with a functional spine unit;
selecting a spinal implant device template from a set of templates based upon the load characteristics;
identifying instantaneous axis of rotation values for the functional spine unit based upon the geometric characteristics;
providing the spinal implant device template and the instantaneous axis of rotation values to a topology optimization engine to generate a spinal implant device model; and
printing the spinal implant device based upon the spinal implant device model.

9. The method of claim 8, wherein receiving geometric characteristics associated with a functional spine unit comprises receiving imaging data associated with the functional spine unit, the imaging data identifying motion of the functional spine unit.

10. The method of claim 8, wherein selecting the spinal implant device template from the set of templates based upon the load characteristics comprises selecting the spinal implant device template having a lattice structure that corresponds to a load response associated with the load characteristics of the functional spine unit.

11. The method of claim 8, wherein generating the spinal implant device model comprises:
identifying instantaneous axis of rotation values of the spinal implant device template by the topology optimization engine;
comparing the instantaneous axis of rotation values of the spinal implant device template with the instantaneous axis of rotation values for the functional spine unit; and
when the instantaneous axis of rotation values of the spinal implant device template lacks a correspondence to the instantaneous axis of rotation values for the functional spine unit:
adjusting a lattice metric of the spinal implant device template, and
generate the spinal implant device model by the topology optimization engine having the adjusted lattice metric of the spinal implant device template.

12. The method of claim 11, further comprising:
identifying instantaneous axis of rotation values of the spinal implant device model by the topology optimization engine;
comparing the instantaneous axis of rotation values of the spinal implant device model with the instantaneous axis of rotation values for the functional spine unit; and
when the instantaneous axis of rotation values of the spinal implant device model lacks a correspondence to the instantaneous axis of rotation values for the functional spine unit:
adjusting a lattice metric of the spinal implant device model, and
generating a subsequent spinal implant device model by the topology optimization engine.

13. The method of claim 8, wherein printing the spinal implant device based upon the spinal implant device model comprises:
printing a first plate from a printing material based upon the spinal implant device model;
printing a second plate from the printing material based upon the spinal implant device model; and
printing a lattice structure from the printing material based upon the spinal implant device model, the lattice structure disposed between the first plate and the second plate, the lattice structure configured to adjust an instantaneous axis of rotation of the spinal implant device between the first plate and the second plate during relative movement of the first plate and the second plate.

14. A spinal implant device production system, comprising:
an additive manufacturing platform having a controller comprising a processor and memory, controller configured to:
receive load characteristics and geometric characteristics associated with a functional spine unit,
select a spinal implant device template from a set of templates based upon the load characteristics,
identify instantaneous axis of rotation values for the functional spine unit based upon the geometric characteristics,
provide the spinal implant device template and the instantaneous axis of rotation values to a topology optimization engine to generate a spinal implant device model, and
an additive manufacturing device disposed in electrical communication with the additive manufacturing platform, the additive manufacturing device configured to print the spinal implant device based upon the spinal implant device model.

15. The spinal implant device production system of claim 14, wherein when receiving geometric characteristics associated with a functional spine unit, the controller is configured to receive imaging data associated with the functional spine unit, the imaging data identifying motion of the functional spine unit.

16. The spinal implant device production system of claim 14, wherein when selecting the spinal implant device template from the set of templates based upon the load characteristics, the controller is configured to select the spinal implant device template having a lattice structure that corresponds to a load response associated with the load characteristics of the functional spine unit.

17. The spinal implant device production system of claim 14, wherein when generating the spinal implant device model, the controller is configured to:
identify instantaneous axis of rotation values of the spinal implant device template by the topology optimization engine;
compare the instantaneous axis of rotation values of the spinal implant device template with the instantaneous axis of rotation values for the functional spine unit; and
when the instantaneous axis of rotation values of the spinal implant device template lacks a correspondence to the instantaneous axis of rotation values for the functional spine unit:
adjust a lattice metric of the spinal implant device template, and
generate the spinal implant device model by the topology optimization engine having the adjusted lattice metric of the spinal implant device template.

18. The spinal implant device production system of claim 17, wherein the controller is further configured to:
identify instantaneous axis of rotation values of the spinal implant device model by the topology optimization engine;
compare the instantaneous axis of rotation values of the spinal implant device model with the instantaneous axis of rotation values for the functional spine unit; and
when the instantaneous axis of rotation values of the spinal implant device model lacks a correspondence to the instantaneous axis of rotation values for the functional spine unit:
adjust a lattice metric of the spinal implant device model, and
generate a subsequent spinal implant device model by the topology optimization engine having the adjusted lattice metric of the spinal implant device template.

19. The spinal implant device production system of claim 14, wherein when printing the spinal implant device based upon the spinal implant device model, the additive manufacturing device is configured to:
print a first plate from a printing material based upon the spinal implant device model;
print a second plate from the printing material based upon the spinal implant device model; and
print a lattice structure from the printing material based upon the spinal implant device model, the lattice structure disposed between the first plate and the second plate, the lattice structure configured to adjust an instantaneous axis of rotation of the spinal implant device between the first plate and the second plate during relative movement of the first plate and the second plate.
